(19) United States
(12) Patent Application Publication
(10) Pub. No.: US 2024/0285539 A1
(43) Pub. Date: Aug. 29, 2024

(54) PROTECTIVE COATING FOR MOISTURE SENSITIVE PHARMACEUTICAL COMPOSITIONS

(71) Applicant: R.P. Scherer Technologies, LLC, Carson City, NV (US)

(72) Inventors: Todd Anthony STUTZMAN, Overland Park, KS (US); Allissa Robin KERNER, Kansas City, MO (US); Joseph Michael SCHNITZ, Kansas City, MO (US); Daniela CIOLOBOC, Kansas City, MO (US)

(73) Assignee: R.P. Scherer Technologies, LLC, Carson City, NV (US)

(21) Appl. No.: 18/567,236

(22) PCT Filed: Jun. 3, 2022
(86) PCT No.: PCT/US2022/072742

§ 371 Date: Dec. 5, 2023

Related U.S. Application Data
(60) Provisional application No. 63/197,880, filed on Jun. 7, 2021.

(51) Int. Cl. A61K 9/28; A61K 31/4545; A61K 33/24

(52) U.S. Cl. CPC A61K 9/2886; A61K 9/2846; A61K 9/288; A61K 31/4545; A61K 33/24

(57) ABSTRACT

Provided are pharmaceutical compositions and methods for preparing pharmaceutical compositions comprising wax coated tablets. Specifically, a wax coating can be applied to a pharmaceutical tablet prior to the film coating process.

### CROSS-REFERENCE TO RELATED APPLICATION

This application is a U.S. national stage application under 35 U.S.C. § 371 of International Application No. PCT/US2022/072742, filed Jun. 3, 2022, which claims priority to U.S. Provisional Application No. 63/197,880, filed Jun. 7, 2021. The entire contents of each priority application is incorporated herein by reference.

### FIELD OF THE DISCLOSURE

This relates to pharmaceutical compositions that include coatings and processes for coating pharmaceutical compositions. Specifically, this relates to wax coatings for pharmaceutical compositions and methods of applying the wax coatings to pharmaceutical compositions.

### BACKGROUND OF THE DISCLOSURE

Pharmaceutical compositions typically include both an active pharmaceutical ingredient as well as one or more inactive ingredients. The active pharmaceutical ingredient (API) can be biologically active and be designed to directly affect a patient's symptoms, diseases, disorders, and/or ailments. The inactive ingredient(s) of a pharmaceutical composition, on the other hand, are pharmaceutically inert and can be used for various purposes including, but not limited to, improving long-term stabilization, filling or diluting a solid formulation, facilitating drug absorption, modifying viscosity of liquid formulations, enhancing solubility, and/or aiding the manufacture of the pharmaceutical composition.

Many pharmaceutical compositions (e.g., dosage forms such as tablets) are coated with an aqueous film coating during the pharmaceutical manufacturing process. However, an aqueous film coating can cause challenges depending on specific APIs as well as the overall formulation of the pharmaceutical composition.

### SUMMARY OF THE DISCLOSURE

Some pharmaceutical compositions include materials that are sensitive to moisture. For example, there are various APIs which are sensitive to moisture and can start to degrade upon introduction of water which can occur during the film coating process. This degradation of API can result in a dosage form that is sub-potent, has a reduced shelf life, and/or compromising of the drug products critical quality attributes (CQAs).

In addition, some of the ingredients of the pharmaceutical compositions can be highly soluble. Thus, when applying a coating to the pharmaceutical composition (e.g., a tablet), the pharmaceutical composition may start to dissolve. This can result in defects in the film coating which may not be acceptable for market.

Applicants have discovered that the pharmaceutical composition (e.g., a tablet) can be first coated with a wax coating prior to initiating the aqueous film coating. Applicants have found that this initial wax coating is not enough to impact the dissolution profile of the pharmaceutical composition tablet, but is enough to provide a protective barrier sufficient to apply the base coat for the film coat without compromising stability or integrity of the core tablet.

Although carnauba wax has historically been used in the film coating process, it has been used after the film coat has been applied to the tablet and used to increase the sheen/gloss of the coating. As such, the wax was used as aesthetic choice, not for a functional application.

In some embodiments, a pharmaceutical composition includes a tablet comprising an active pharmaceutical ingredient (API); a first coating on a surface of the tablet comprising wax; and a second coating on a surface of the first coating, wherein the second coating comprises polyvinyl alcohol. In some embodiments, the wax comprises carnauba wax. In some embodiments, the pharmaceutical composition comprises 0.5-1.0% w/w wax. In some embodiments, the first coating consists of wax. In some embodiments, the second coating is an OPADRY® moisture barrier film coating. In some embodiments, the pharmaceutical composition includes a third coating on a surface of the second coating, wherein the third coating comprises an acrylic acid and/or an acrylate. In some embodiments, the third coating is an ACRYL-EZE® enteric coating. In some embodiments, the API comprises ALXN1840 or AKST4290. In some embodiments, the tablet comprises sodium bicarbonate.

In some embodiments, a method for preparing a pharmaceutical composition includes compressing a tablet comprising an active pharmaceutical ingredient (API); coating a surface of the tablet with a first coating comprising wax; and coating a surface of the first coating with a second coating comprising polyvinyl alcohol. In some embodiments, the coating the surface of the tablet with the first coating comprises coating the surface of the tablet such that the weight gain of the tablet with the first coating is 0.5-1.0% from the first coating. In some embodiments, the coating the surface of the first coating comprises coating the surface of the first coating such that the weight gain of the tablet and first coating is 1-30% from the second coating. In some embodiments, the weight gain of the tablet and first coating from the second coating is 15-25%. In some embodiments, the wax comprises carnauba wax. In some embodiments, the first coating consists of wax. In some embodiments, the second coating is an OPADRY® moisture barrier film coating. In some embodiments, the method includes coating a surface of the second coating with a third coating comprising an acrylic acid and/or an acrylate. In some embodiments, the third coating is an ACRYL-EZE® enteric coating. In some embodiments, the API comprises ALXN1840 or AKST4290. In some embodiments, the tablet comprises sodium bicarbonate.

Additional advantages will be readily apparent to those skilled in the art from the following detailed description. The examples and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE DRAWINGS

FIG. 1A illustrates tablets coated with a film coating without a wax coating, according to some embodiments disclosed herein.

FIG. 1B illustrates additional tablets coated with a film coating without a wax coating, according to some embodiments disclosed herein.

FIG. 2A illustrates a side view of an uncoated tablet and a tablet coated with a film and enteric coating, according to some embodiments disclosed herein.

FIG. 2B illustrates a top view of an uncoated tablet and a tablet coated with a film and enteric coating, according to some embodiments disclosed herein.

### DETAILED DESCRIPTION OF THE DISCLOSURE

Described herein are exemplary embodiments of pharmaceutical compositions and methods for coating pharmaceutical compositions to provide a protective barrier to the pharmaceutical compositions and/or API. Prior to aqueous film coating, a pharmaceutical composition can be coated with a wax coating that does not compromise the stability or integrity of the pharmaceutical composition.

The pharmaceutical composition can include a dosage form such as a tablet comprising an active pharmaceutical ingredient as well as a plurality of other ingredients that make up the tablet. The pharmaceutical composition can also include a wax coating on a surface of the tablet and any subsequent coatings to the tablet. As such, as used herein below, a pharmaceutical composition can refer to a tablet and all coatings that are applied to the tablet.

The tablet can be made by a variety of methods such as wet granulation, dry granulation, or direct compression. A wet granulation process typically includes: (1) mixing of the API with other ingredients to form a powder; (2) preparing of binder solution; (3) mixing of binder solution with the powder; (4) screening the dampened powder into pellets/granules; (5) drying the pellets/granules; (6) sizing the granulation by dry screening; (7) mixing dried pellets/granules with lubricant and disintegrant; and/or (8) compressing of pellets/granules into tablets. A dry granulation process typically includes: (1) mixing of the API with other ingredients to form a powder; (2) compressing the powder into slugs; (3) milling/sieving the slugs; (4) mixing the slugs with lubricant and disintegrant; and/or (5) compressing into tablets. A direct compression process typically includes: (1) mixing of the API with other ingredients (including disintegrants and lubricants); and (2) compressing the powder into tablets.

Any API may be used with the pharmaceutical compositions disclosed herein. One of ordinary skill in the art would understand that for various reasons such as stability, compatibility with other ingredients, desired drug release profile, certain active ingredients and/or APIs are more desirable for formulation into a dosage form. In some embodiments, the API may be an active pharmaceutical ingredient for the treatment of human or veterinary diseases. The API can be the component that the solid dosage form is used to deliver. APIs may be one or more of antibacterial agents, antifungal agents, antiprotozoal agents, antiviral agents, labor-inducing agents, spermicidal agents, prostaglandins, steroids and microbicides, proteins/peptides and vaccine antigens.

APIs may include pharmaceutical ingredients as well as other types of active ingredients that may be ingested, such as vitamins and dietary supplements. Suitable APIs include, without limitation: analgesics and anti-inflammatory agents (e.g., ibuprofen), antacids, anthelmintics, anti-arrhythmic agents, anti-bacterial agents, anti-coagulants, anti-anxiety anti-depressants, anti-diabetics, anti-diarrhoeals, anti-epileptics, anti-fungal agents, anti-gout agents, anti-hypertensive agents, anti-malarials, anti-migraine agents, anti-muscarinic agents, anti-neoplastic agents and immunosuppressants, anti-protazoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuro muscular agents, nitrates and anti-anginal agents, nutritional agents, opioid analgesics, oral vaccines, proteins, peptides and recombinant drugs, sex hormones and contraceptives, spermicides, stimulants, smoking cessation products and combinations thereof.

The API may be a single active pharmaceutical ingredient, such as a single chemical entity, or it may be a mixture of several active pharmaceutical ingredients. The active pharmaceutical ingredient may be of any of the many categories of active pharmaceutical ingredients. The active pharmaceutical ingredient may be selected from, but is not limited to, the group consisting of acyclovir, fluconazole, progesterone and derivatives thereof, nonoxylenol-9, terbutaline, lidocaine, testosterone and derivatives, dinoprostone, lactobacillus, estrogen and derivatives, naphthalene2-sulfonate, lesmitidan, doxycycline, droxidopa, sapropterin, butoconazole, clindamycin nitrate/phosphate, neomycine sulfate, polymyxin sulfate, nystatin, clotrimazole, dextrin sulphate, glyminox, miconazole nitrate, benzalkonium chloride, sodium lauryl sulphate, tenofovir, insulin, calcitonin, danazol, ibuprofen, acetaminophen, cefpodoxime proxetil, desloratadine, dextromethorphan, diphenhydramine hydrochloride, vitamins and/or minerals, adipic acid, ascorbic acid, macrolide antibiotics, NS-AIDS, cefuroxime axetil, amobarbital, ciprofloxacin hydrochloride, sildenafil citrate, pinaverium bromide, propantheline bromide, triprolidine Hcl, dimenhydrinate, cefeanel daloxate HCl, Enoxacin, Sparfloxacin, aspirin, famotidine, amoxycilin trihydrate, morphine HCl, amiprilose HCl, terfenadine, beclamide, clarithromycin, roxithromycin, nizatidine, cetraxate HCl, ciprofloxacin, bifemelene HCl, Cefuroxime axetil, pirienzepine and/or oxyburynin, diclofenac, nicorandil, levofloxacin, acriflavine, leuprorelin acetate, metronidazole, benzydamine hydrochloride, chloramphenicol, oxybutynin, ethinyl estradiol, prostaglandins, insulin, calcitonin and combinations thereof. The active pharmaceutical ingredient may also be vaccine antigen such as those for the treatment of Hepatitis B, HIV, HPV, Chlamydia, gonococcal infections. In some embodiments, the API can be ALXN1840 or AKST4290.

APIs may include salts, esters, hydrates, solvates and derivatives of any of the foregoing active ingredients. Suitable derivatives are those that are known to skilled persons to possess the same activity as the active ingredient though the activity level may be lower or higher. APIs may also include any active ingredient that is incompatible with oral delivery methods or compositions.

When present, an API is employed in the formulation in an effective amount that is necessary to provide the dosage required, typically for producing at least one physiological effect as established by clinical studies. One of ordinary skill in the art can readily determine an appropriate amount of active ingredient to include in the dosage form (e.g., tablet) made according to the present disclosure.

In some embodiments, the tablet of the pharmaceutical composition may comprise about 1-20 wt. %, about 1-15 wt. %, about 5-10 wt. %, about 7-9 wt. %, or about 8-9 wt. % API. In some embodiments, the tablet of the pharmaceutical composition may comprise at least about 1 wt. %, at least about 5 wt. %, at least about 8 wt. %, at least about 10 wt. %, at least about 15 wt. %, or at least about 20 wt. % API. In some embodiments, the tablet of the pharmaceutical composition may comprise at most about 30 wt. %, at most about 25 wt. %, at most about 20 wt. %, at most about 15 wt. %, at most about 10 wt. %, or at most about 9 wt. % API. In some embodiments, the pharmaceutical composition (including all coatings) may comprise about 1-20 wt. %, about 1-15 wt. %, about 2-8 wt. %, about 3-8 wt. %, or about 4-6 wt. % API. In some embodiments, the pharmaceutical composition (including all coatings) may comprise at least 1 wt. % API, at least 2 wt. %, API, at least 3 wt. %, API, at least 4 wt. % API, or at least 5 wt. % API. In some embodiments, the pharmaceutical composition (including all coatings) may comprise at most about 20 wt. %, at most about 15 wt. %, or at most about 10 wt. % API.

In some embodiments, the tablet of the pharmaceutical composition may comprise about 50-90 wt. %, about 60-80 wt. %, about 60-75 wt. %, about 60-70 wt. %, or about 65-70 wt. % API. In some embodiments, the tablet of the pharmaceutical composition includes at least 50 wt. %, at least 55 wt. %, at least 60 wt. %, or at least 65 wt. % API. In some embodiments, the tablet of the pharmaceutical composition includes at most 90 wt. %, at most 85 wt. %, at most 80 wt. %, at most 75 wt. %, or at most 70 wt. % API. In some embodiments, pharmaceutical composition (including all coatings) may comprise about 50-90 wt. %, about 60-80 wt. %, about 60-75 wt. %, about 60-70 wt. %, or about 63-67 wt. % API. In some embodiments, the pharmaceutical composition (including all coatings) includes at least 50 wt. %, at least 55 wt. %, at least 60 wt. %, or at least 64 wt. % API. In some embodiments, the pharmaceutical composition (including all coatings) includes at most 90 wt. %, at most 85 wt. %, at most 80 wt. %, at most 75 wt. %, or at most 70 wt. % API.

In some embodiments, the tablet makes up about 50-99 wt. %, about 55-99 wt. %, or about 60-99 wt. % of the pharmaceutical composition. In some embodiments, the tablet makes up at least 50 wt. %, at least 55 wt. %, at least 60 wt. %, at least 65 wt. %, at least 70 wt. %, at least 75 wt. %, at least 80 wt. %, at least 85 wt. %, at least 90 wt. %, or at least 95 wt. % of the pharmaceutical composition. In some embodiments, the tablet makes up at most 99 wt. %, at most 98 wt. %, at most 97 wt. %, at most 95 wt. %, at most 90 wt. %, at most 85 wt. %, at most 80 wt. %, at most 75 wt. %, at most 70 wt. %, or at most 65 wt. % of the pharmaceutical composition.

In some embodiments, the tablet of the pharmaceutical composition can include an API stabilizer. In some embodiments, the API stabilizer can stabilize the API by maintaining the pH to a certain level. For example, an API stabilizer may prevent an API from becoming acidic. In some embodiments, the API stabilizer can be sodium bicarbonate, citric acid, fumaric acid, malic acid, potassium bicarbonate, sodium carbonate, sodium citrate dehydrate, and/or tartaric acid. In some embodiments, the tablet of the pharmaceutical composition can include about 15-35 wt. %, about 20-30 wt. %, or about 23-27 wt. % API stabilizer. In some embodiments, the tablet of the pharmaceutical composition can include at least about 15 wt. %, at least about 20 wt. %, or at least about 25 wt. % API stabilizer. In some embodiments, the tablet of the pharmaceutical composition can include at most about 40 wt. %, at most about 35 wt. %, or at most about 30 wt. % API stabilizer. In some embodiments, the pharmaceutical composition (including all coatings) includes about 5-25 wt. %, about 10-20 wt. %, or about 13-17 wt. % API stabilizer. In some embodiments, the pharmaceutical composition (including all coatings) includes at least about 5 wt. %, at least about 10 wt. %, or at least about 15 wt. % API stabilizer. In some embodiments, the pharmaceutical composition (including all coatings) includes at most about 30 wt. %, at most about 25 wt. %, or at most about 20 wt. % API stabilizer.

In some embodiments, the tablet of the pharmaceutical composition includes a binder. In some embodiments, the binder can also be a diluent. In some embodiments, the binder can be cellulose (e.g., microcrystalline cellulose (e.g., Avicel PH 112)), dextrate, starch, calcium carbonate, and/or dextrose. In some embodiments, the tablet of the pharmaceutical composition can include about 55-75 wt. %, about 60-70 wt. %, or about 63-67 wt. % binder. In some embodiments, the tablet of the pharmaceutical composition can include at least about 55 wt. %, at least about 60 wt. %, or at least about 65 wt. % binder. In some embodiments, the tablet of the pharmaceutical composition can include at most about 80 wt. %, at most about 75 wt. %, or at most about 70 wt. % binder. In some embodiments, the pharmaceutical composition (including all coatings) includes about 30-50 wt. %, about 35-45 wt. %, or about 38-42 wt. % binder. In some embodiments, the pharmaceutical composition (including all coatings) includes at least about 30 wt. %, at least about 35 wt. %, or at least about 40 wt. % binder. In some embodiments, the pharmaceutical composition (including all coatings) includes at most about 55 wt. %, at most about 50 wt. %, or at most about 45 wt. % binder.

In some embodiments, the tablet of the pharmaceutical composition can include about 1-10 wt. %, about 2-8 wt. %, or about 4-8 wt. % binder. In some embodiments, the tablet of the pharmaceutical composition can include at least about 1 wt. %, at least about 2 wt. %, or at least about 5 wt. % binder. In some embodiments, the tablet of the pharmaceutical composition can include at most about 15 wt. %, at most about 10 wt. %, or at most about 8 wt. % binder. In some embodiments, the pharmaceutical composition (including all coatings) includes about 1-10 wt. %, about 2-8 wt. %, or about 4-8 wt. % binder. In some embodiments, the pharmaceutical composition (including all coatings) includes at least about 1 wt. %, at least about 3 wt. %, or at least about 5 wt. % binder. In some embodiments, the pharmaceutical composition (including all coatings) includes at most about 15 wt. %, at most about 10 wt. %, or at most about 8 wt. % binder.

In some embodiments, the tablet of the pharmaceutical composition can include about 1-25 wt. %, about 5-20 wt. %, or about 10-15 wt. % a diluent. In some embodiments, the diluent is a water soluble diluent. In some embodiments, the diluent can be lactose (e.g., lactose anhydrous), mannitol, maltose, sorbitol, various grades of MCC, dicalcium phosphate, and/or starch (e.g., pregelatinized starch). In some embodiments, the tablet of the pharmaceutical composition can include at least about 1 wt. %, at least about 5 wt. %, or at least about 10 wt. % diluent. In some embodiments, the tablet of the pharmaceutical composition can include at most about 25 wt. %, at most about 20 wt. %, or at most about 15 wt. % diluent. In some embodiments, the pharmaceutical composition (including all coatings) includes about 1-25 wt. %, about 5-20 wt. %, or about 10-15 wt. % diluent. In some embodiments, the pharmaceutical composition (including all coatings) includes at least about 1 wt. %, at least about 5 wt. %, or at least about 10 wt. % diluent. In some embodiments, the pharmaceutical composition (including all coatings) includes at most about 25 wt. %, at most about 20 wt. %, or at most about 15 wt. % diluent.

In some embodiments, the tablet of the pharmaceutical composition can include about 0.1-1 wt. %, about 0.1-0.5 wt. %, or about 0.2-0.3 wt. % a glidant. In some embodiments, the glidant can be a colloidal silicon dioxide, magnesium carbonate, talc, and/or calcium silicate. In some embodiments, the colloidal silicon dioxide can be aerosil 200. In some embodiments, the tablet of the pharmaceutical composition can include at least about 0.1 wt. %, at least about 0.15 wt. %, or at least about 0.2 wt. % glidant. In some embodiments, the tablet of the pharmaceutical composition can include at most about 1 wt. %, at most about 0.5 wt. %, or at most about 0.3 wt. % glidant. In some embodiments, the pharmaceutical composition (including all coatings) includes about 0.1-1 wt. %, about 0.1-0.5 wt. %, or about 0.2-0.3 wt. % glidant. In some embodiments, the pharmaceutical composition (including all coatings) includes at least about 0.1 wt. %, at least about 0.15 wt. %, or at least about 0.2 wt. % glidant. In some embodiments, the pharmaceutical composition (including all coatings) includes at most about 1 wt. %, at most about 0.5 wt. %, or at most about 0.3 wt. % glidant.

In some embodiments, the tablet can include intragranular ingredients as well as extragranular ingredients. The intragranular ingredients can be those ingredients that are roller compacted and/or wet compacted. The extragranular ingredients can be those ingredients that are added after granulation.

In some embodiments, the tablet of the pharmaceutical composition can include about 1-10 wt. %, about 2-8 wt. %, or about 3-7 wt. % a superdisintegrant (intragranular). In some embodiments, the superdisintegrant (intragranular) can be sodium croscarmellose (intragranular) (e.g., Croscarmellose Sodium NF. Ph. Eur., JP (Ac-Di-Sol)), sodium starch glycolate (intragranular), and/or crospovidone (intragranular). In some embodiments, the tablet of the pharmaceutical composition can include at least about 1 wt. %, at least about 2 wt. %, or at least about 3 wt. % superdisintegrant (intragranular). In some embodiments, the tablet of the pharmaceutical composition can include at most about 10 wt. %, at most about 8 wt. %, or at most about 6 wt. % superdisintegrant (intragranular). In some embodiments, the pharmaceutical composition (including all coatings) includes about 1-10 wt. %, about 2-8 wt. %, or about 3-7 wt. % superdisintegrant (intragranular). In some embodiments, the pharmaceutical composition (including all coatings) includes at least about 1 wt. %, at least about 2 wt. %, or at least about 3 wt. % superdisintegrant (intragranular). In some embodiments, the pharmaceutical composition (including all coatings) includes at most about 10 wt. %, at most about 7 wt. %, or at most about 5 wt. % superdisintegrant (intragranular).

In some embodiments, the tablet of the pharmaceutical composition can include about 1-10 wt. %, about 2-8 wt. %, or about 3-7 wt. % superdisintegrant (extragranular) In some embodiments, the superdisintegrant (extragranular) can be sodium croscarmellose (extragranular) (e.g., Croscarmellose Sodium NF. Ph. Eur., JP (Ac-Di-Sol)), sodium starch glycolate (extragranular), or crospovidone (extragranular). In some embodiments, the tablet of the pharmaceutical composition can include at least about 1 wt. %, at least about 2 wt. %, or at least about 3 wt. % superdisintegrant (extragranular). In some embodiments, the tablet of the pharmaceutical composition can include at most about 10 wt. %, at most about 8 wt. %, or at most about 6 wt. % superdisintegrant (extragranular). In some embodiments, the pharmaceutical composition (including all coatings) includes about 1-10 wt. %, about 2-8 wt. %, or about 3-7 wt. % superdisintegrant (extragranular). In some embodiments, the pharmaceutical composition (including all coatings) includes at least about 1 wt. %, at least about 2 wt. %, or at least about 3 wt. % superdisintegrant (extragranular). In some embodiments, the pharmaceutical composition (including all coatings) includes at most about 10 wt. %, at most about 7 wt. %, or at most about 5 wt. % superdisintegrant (extragranular).

In some embodiments, the tablet of the pharmaceutical composition can include about 0.1-3 wt. %, about 0.2-2.5 wt. %, or about 0.25-1.5 wt. % lubricant (intragranular). In some embodiments, the lubricant can be sodium stearyl fumarate (intragranular), stearic acid (intragranular), compritol (intragranular), calcium stearate (intragranular), and/or magnesium stearate (intragranular). In some embodiments, the tablet of the pharmaceutical composition can include at least about 0.1 wt. %, at least about 0.2 wt. %, at least about 0.25 wt. %, at least about 0.5 wt. %, at least about 1 wt. %, or at least about 1.5 wt. % lubricant (intragranular). In some embodiments, the tablet of the pharmaceutical composition can include at most about 3 wt. %, at most about 2 wt. %, at most about 1.5 wt. %, at most about 1 wt. %, or at most about 0.5 wt. % lubricant (intragranular). In some embodiments, the pharmaceutical composition (including all coatings) includes about 0.1-3 wt. %, about 0.1-2.5 wt. %, or about 0.15-1.5 wt. % lubricant (intragranular). In some embodiments, the pharmaceutical composition (including all coatings) includes at least about 0.1 wt. %, at least about 0.15 wt. %, at least about 0.2 wt. %, at least about 0.3 wt. %, at least about 0.5 wt. %, or at least about 1 wt. % lubricant (intragranular). In some embodiments, the pharmaceutical composition (including all coatings) includes at most about 3 wt. %, at most about 2 wt. %, at most about 1.5 wt. %, at most about 1 wt. %, or at most about 0.5 wt. % lubricant (intragranular).

In some embodiments, the tablet of the pharmaceutical composition can include about 0.1-3 wt. %, about 0.2-2.5 wt. %, or about 0.25-1.5 wt. % lubricant (extragranular) In some embodiments, the lubricant (extragranular) can be sodium stearyl fumarate (extragranular), stearic acid (extragranular), compritol (extragranular), calcium stearate (intragranular), and/or magnesium stearate (extragranular). In some embodiments, the tablet of the pharmaceutical composition can include at least about 0.1 wt. %, at least about 0.2 wt. %, at least about 0.25 wt. %, at least about 0.5 wt. %, at least about 1 wt. %, or at least about 1.5 wt. % lubricant (extragranular). In some embodiments, the tablet of the pharmaceutical composition can include at most about 3 wt. %, at most about 2 wt. %, at most about 1.5 wt. %, at most about 1 wt. %, or at most about 0.5 wt. % lubricant (extragranular). In some embodiments, the pharmaceutical composition (including all coatings) includes about 0.1-3 wt. %, about 0.1-2.5 wt. %, or about 0.15-1.5 wt. % lubricant (extragranular). In some embodiments, the pharmaceutical composition (including all coatings) includes at least about 0.1 wt. %, at least about 0.15 wt. %, at least about 0.2 wt. %, at least about 0.3 wt. %, at least about 0.5 wt. %, or at least about 1 wt. % lubricant (extragranular). In some embodiments, the pharmaceutical composition (including all coatings) includes at most about 3 wt. %, at most about 2 wt. %, at most about 1.5 wt. %, at most about 1 wt. %, or at most about 0.5 wt. % lubricant (extragranular).

In some embodiments, the tablet of the pharmaceutical composition can include about 0.1-2 wt. %, about 0.3-1.5 wt. %, or about 0.5-1 wt. % a surfactant (intragranular). In some embodiments, the surfactant (intragranular) can be sodium lauryl sulfate (intragranular), carbowax PEG 3350 Sentry powder (or other PEG variants) (intragranular), polaxamer (various grades) (intragranular), and/or polysorbates (various grades) (intragranular). In some embodiments, the tablet of the pharmaceutical composition can include at least about 0.1 wt. %, at least about 0.2 wt. %, at least about 0.3 wt. %, at least about 0.5 wt. %, at least about 0.7 wt. %, or at least about 0.75 wt. % surfactant (intragranular). In some embodiments, the tablet of the pharmaceutical composition can include at most about 2 wt. %, at most about 1.5 wt. %, at most about 1 wt. %, at most about 0.9 wt. %, or at most about 0.8 wt. % surfactant (intragranular). In some embodiments, the pharmaceutical composition (including all coatings) includes about 0.1-2 wt. %, about 0.3-1.5 wt. %, or about 0.5-1 wt. % surfactant (intragranular). In some embodiments, the pharmaceutical composition (including all coatings) includes at least about 0.1 wt. %, at least about 0.2 wt. %, at least about 0.3 wt. %, at least about 0.5 wt. %, at least about 0.7 wt. %, or at least about 0.75 wt. % surfactant (intragranular). In some embodiments, the pharmaceutical composition (including all coatings) includes at most about 2 wt. %, at most about 1.5 wt. %, at most about 1 wt. %, at most about 0.9 wt. %, or at most about 0.8 wt. % surfactant (intragranular).

In some embodiments, the tablet of the pharmaceutical composition can include about 0.1-2 wt. %, about 0.3-1.5 wt. %, or about 0.5-1 wt. % surfactant (extragranular). In some embodiments, the surfactant (extragranular) can be sodium lauryl sulfate (extragranular), carbowax PEG 3350 Sentry powder (or other PEG variants) (extragranular), polaxamer (various grades) (extragranular), and/or polysorbates (various grades) (extragranular). In some embodiments, the tablet of the pharmaceutical composition can include at least about 0.1 wt. %, at least about 0.2 wt. %, at least about 0.3 wt. %, at least about 0.5 wt. %, at least about 0.7 wt. %, or at least about 0.75 wt. % surfactant (extragranular). In some embodiments, the tablet of the pharmaceutical composition can include at most about 2 wt. %, at most about 1.5 wt. %, at most about 1 wt. %, at most about 0.9 wt. %, or at most about 0.8 wt. % surfactant (extragranular). In some embodiments, the pharmaceutical composition (including all coatings) includes about 0.1-2 wt. %, about 0.3-1.5 wt. %, or about 0.5-1 wt. % surfactant (extragranular). In some embodiments, the pharmaceutical composition (including all coatings) includes at least about 0.1 wt. %, at least about 0.2 wt. %, at least about 0.3 wt. %, at least about 0.5 wt. %, at least about 0.7 wt. %, or at least about 0.75 wt. % surfactant (extragranular). In some embodiments, the pharmaceutical composition (including all coatings) includes at most about 2 wt. %, at most about 1.5 wt. %, at most about 1 wt. %, at most about 0.9 wt. %, or at most about 0.8 wt. % surfactant (extragranular).

In some embodiments, the tablet of the pharmaceutical composition can include about 0.1-1 wt. %, about 0.1-0.5 wt. %, or about 0.2-0.3 wt. % a second lubricant (intragranular). In some embodiments, the second lubricant (intragranular) can be any lubricant disclosed herein. In some embodiments, the tablet of the pharmaceutical composition can include at least about 0.1 wt. %, at least about 0.15 wt. %, or at least about 0.2 wt. % second lubricant (intragranular). In some embodiments, the tablet of the pharmaceutical composition can include at most about 1 wt. %, at most about 0.5 wt. %, or at most about 0.3 wt. % second lubricant (intragranular). In some embodiments, the pharmaceutical composition (including all coatings) includes about 0.1-1 wt. %, about 0.1-0.5 wt. %, or about 0.2-0.3 wt. % second lubricant (intragranular). In some embodiments, the pharmaceutical composition (including all coatings) includes at least about 0.1 wt. %, at least about 0.15 wt. %, or at least about 0.2 wt. % second lubricant (intragranular). In some embodiments, the pharmaceutical composition (including all coatings) includes at most about 1 wt. %, at most about 0.5 wt. %, or at most about 0.3 wt. % second lubricant (intragranular).

In some embodiments, the tablet of the pharmaceutical composition can include about 0.1-1 wt. %, about 0.1-0.5 wt. %, or about 0.2-0.3 wt. % second lubricant (extragranular). In some embodiments, the second lubricant (extragranular) can be any lubricant disclosed herein. In some embodiments, the tablet of the pharmaceutical composition can include at least about 0.1 wt. %, at least about 0.15 wt. %, or at least about 0.2 wt. % second lubricant (extragranular). In some embodiments, the tablet of the pharmaceutical composition can include at most about 1 wt. %, at most about 0.5 wt. %, or at most about 0.3 wt. % second lubricant (extragranular). In some embodiments, the pharmaceutical composition (including all coatings) includes about 0.1-1 wt. %, about 0.1-0.5 wt. %, or about 0.2-0.3 wt. % second lubricant (extragranular). In some embodiments, the pharmaceutical composition (including all coatings) includes at least about 0.1 wt. %, at least about 0.15 wt. %, or at least about 0.2 wt. % second lubricant (extragranular). In some embodiments, the pharmaceutical composition (including all coatings) includes at most about 1 wt. %, at most about 0.5 wt. %, or at most about 0.3 wt. % second lubricant (extragranular).

As explained above, the tablets having the compositions disclosed above can be formed from either wet granulation, dry granulation, or direct compression. Once the tablet is formed, the tablet can be coated with a first coating directly covering the surface of the tablet. This first coating can be a wax coating. The wax of the wax coating can be carnauba wax, beewax, and other waxes. In some embodiments, the coating can be accomplished by a Vector Coater, Compu-Lab 24, Compu-Lab 35, and/or Accela Cota Coaters equipped with various size pans (e.g., 15″, 19″ pans).

As mentioned above, Applicant has discovered that a small amount of wax can provide a protective barrier sufficient to apply the film coat without impacting the dissolution profile of the tablet or compromising the stability/integrity of the tablet. In some embodiments, the wax coating can be applied as a 0.5-1.0% weight gain to the tablet itself. For example, if the tablet weighs 15 mg, a 1% weight gain adds 0.15 mg wax to the tablet. In some embodiments, the wax coating can be applied as about 0.1-2% weight gain, about 0.3-1.5% weight gain, or about 0.5-1.0% weight gain to the tablet. In some embodiments, the amount of wax in the tablet plus the wax coating can be about 0.1-2 wt. %, about 0.3-1.5 wt. %, or about 0.5-1 wt. %. In some embodiments, the amount of wax in the tablet plus the wax coating can be at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, or at least about 0.8 wt. %. In some embodiments, the amount of wax in the tablet plus the wax coating can be at most about 2 wt. %, at most about 1.5 wt. %, at most about 1 wt. %, at most about 0.9 wt. %, or at most about 0.8 wt. %. In some embodiments, the amount of wax in the pharmaceutical composition (including all coatings) includes about 0.1-2 wt. %, about 0.3-1.5 wt. %, or about 0.5-1 wt. %. In some embodiments, the amount of wax in the pharmaceutical composition (including all coatings) can be at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, or at least about 0.8 wt. %. In some embodiments, the amount of wax in the pharmaceutical composition (including all coatings) can be at most about 2 wt. %, at most about 1.5 wt. %, at most about 1 wt. %, at most about 0.9 wt. %, or at most about 0.8 wt. %.

In some embodiments, the wax coating can be applied to the tablets by the following process. After the tablets have been sufficiently heated (e.g., once the bed pan achieves a temperature of greater than about 20° C., greater than about 25° C., greater than about 30° C., greater than about 35° C., greater than about 40° C., or greater than about 45° C.; and less than about 50° C., less than about 45° C., less than about 40° C., less than about 35° C., less than about 30° C., or less than about 25° C.), the inlet and exhaust fan can be turned off and a pre-determined appropriate aliquot of wax can be slowly (e.g., over approximately 2-5 minutes) added to the pan sprinkling on top of the core tablets with the pan rotating. In some embodiments, a waxed paper sheet can be placed under the pan. In some embodiments, the wax from the waxed paper can be recovered and sprinkled on top of the core tablets. The pan speed can be adjusted based upon visual observation of the tablet bed. The tablets can be tumbled with wax for about 5-10 minutes at about 3 RPM. After the 5-10 minutes, the exhaust fan can be turned on and run for an additional 3-7 minutes. The inlet fan can also be turned on and run for an additional 3-7 minutes. Next, the wax coated tablets can be removed for further processing.

After the tablets have been coated with a layer of wax, they can be film coated. Film coating can involve the deposition of a film forming polymeric onto the wax coating. Typically, the film coating formulation can include a polymer (e.g., polyvinyl alcohol) solubilized in a suitable solvent together with other additives such as plasticizers and pigments/colorants. Examples of film coatings can include OPADRY® moisture barrier film coatings such as OPADRY® AMBII, OPADRY® 200, OPADRY® II, and/or OPADRY® QX (any Opadry line). The film coating formulation can be sprayed onto a rotating or fluidized tablet bed. The drying conditions for the film coating process can remove the solvent, leaving a thin layer of coating material around the wax tablet.

In some embodiments, the film coating can be prepared as a solids suspension and applied to the wax coated tablet at a weight gain of about 1-30%, about 3-25%, or about 3-20%. In some embodiments, the solids suspension can be a 10-30% solids suspension, 15-25% solids suspension or a 15-20% solids suspension for the film coating. In some embodiments, the amount of film coating of the pharmaceutical composition (including all coatings) is at least about 1 wt. %, at least 2 wt. %, at least about 3 wt. %, at least about 5 wt. %, at least about 8 wt. %, at least about 10 wt. %, or at least about 12 wt. %. In some embodiments, the amount of film coating of the pharmaceutical composition (including all coatings) is at most about 25 wt. %, at most about 20 wt. %, at most about 15 wt. %, at most about 10 wt. %, at most about 8 wt. %, at most about 5 wt. %, or at most about 3 wt. %.

After the tablets have been coated with a film coating, the film coating layer can be coated with an enteric coating such as an ACRYL-EZE® enteric coating. The enteric coating can include an acrylic acid and/or an acrylate. The enteric coating formulation can be sprayed onto a rotating or fluidized tablet bed. The drying conditions for the enteric coating process can remove the solvent, leaving a thin layer of coating material around the wax and film coated tablet.

In some embodiments, the enteric coating can be prepared as a solids suspension and applied to the wax and film coated tablet at a weight gain of about 20-50%, about 25-45%, or about 30-40%. In some embodiments, the solids suspension can be a 10-30% solids suspension, 15-25% solids suspension or a 15-20% solids suspension for the enteric coating. In some embodiments, the amount of enteric coating of the pharmaceutical composition (including all coatings) is at least about 10 wt. %, at least about 15 wt. %, at least about 20 wt. %, or at least about 25 wt. %. In some embodiments, the amount of enteric coating of the pharmaceutical composition (including all coatings) is at most about 50 wt. %, at most about 45 wt. %, at most about 40 wt. %, at most about 35 wt. %, or at most about 30 wt. %.

The preventive wax coatings described herein can be used across multiple moisture sensitive APIs, or highly soluble, quick dissolving, effervescent tablets. The wax coating can be applied to tablets prior to applying the aqueous film coats to provide this protective wax coating layer.

### EXAMPLES

### Example 1: AKST-4290 as an API

The Alkahest compound AKST-2490 is a highly soluble API that can degrade with moisture. Applicant has been able to prevent degradation utilizing the wax coating process described herein. This technique reduced the coating time and limited water uptake of core tablets improving impurity profile at T-0.

Specifically, the wax was applied as a sub-coat to the core tablets. In the case of AKST-4290, one of the purposes of this wax coat was to protect the API in the tablets from moisture. This API is highly hygroscopic and when exposed to a relative humidity over 40% (even for a couple of minutes) undergoes both physical and chemical changes. Although the relative humidity can be controlled during the upstream process of creating the tablet, this cannot be achieved in a coating process with aqueous solutions.

For this example, the film coating applied was OPADRY AMBII series formulated to provide an oxygen and humidity barrier. However, the film coating formulation is applied as a 15% solids aqueous solution and therefore the core tablets are exposed to moisture during film coating. However, Applicants discovered that a thin wax layer can be sufficient to protect the core tablets during film coating. The following is the compositions of the tablet, first wax coating, and second film coating:

| | Components | | |
|---|---|---|---|
| Intragranular | AKST-4290 | 66.50 | |
| components | Avicel PH-112 | 6.75 | |
| | Lactose Anhydrous | 12.50 | |
| | Starch 1500 | | |
| | Croscarmellose Sodium NF. Ph. Eur., | 4.00 | |
| | JP (Ac-Di-Sol) | | |
| | Aerosil 200 | 0.25 | |
| | Sodium Lauryl Sulfate | 0.75 | |
| | Magnesium Stearate NF/EP (Non- | 0.25 | |
| | Bovine #5712) | | |
| | Sodium Stearyl Fumarate | 1.50 | |
| Extragranular | Croscarmellose Sodium NF. Ph. Eur., | 5.00 | |
| components | JP (Ac-Di-Sol) | | |
| | Sodium Lauryl Sulfate | 0.75 | |
| | Magnesium Stearate NF/EP (Non- | 0.25 | |
| | Bovine #5712) | | |
| | Sodium Stearyl Fumarate | 1.50 | |
| | Total [%] | 100.00 | |
| | Core Tablet weight [mg] | 687.12 | mg |
| Coating | Carnuaba Wax NF#1 | 0.5% | |
| | Opadry AMB II Pink | 3.0% | |
| | Total coated tablet [mg] | 711.25 | g |

The 0.5% addition of wax corresponds to a 0.5% weight gain on the core tablet. The 3.0% addition of the OPADRY® coating corresponds to a 3% weight gain on the wax coated tablet.

The wax coating process was as follows: After tablets were sufficiently heated, the inlet and exhaust were turned off, a waxed paper was placed under the pan, and carnauba wax was slowly (over approximately 2-5 minutes) added to the pan sprinkling on top of the core tablets with the pan rotating. The carnauba wax was recovered from the waxed paper and sprinkled on top of the core tablets. Pan speed was adjusted based upon visual observation of the tablet bed. The tablets were tumbled with wax for 5-10 minutes at approximately 3 RPM. After the 5-10 minutes, the exhaust was turned on and run for an additional 3-5 minutes. The inlet was turned on and run for an additional 3-5 minutes. The tablets could then be removed but the temperature of the exhaust was ensured to reach 46° C. (±2° C.) prior to proceed with film coating.

Preliminary results show that the wax and OPADRY® AMBII coated tablets have a lower water content (measured by Karl Fischer analysis) than the ones coated with OPADRY® alone as shown in the following table:

| | At Start (%) | | | 4W4 weeks at 40° C., 75% RH (%) | | |
|---|---|---|---|---|---|---|
| | Rep 1 | Rep 2 | Average | Rep 1 | Rep 2 | Average |
| Opadry II | 2.5913 | 2.603 | 2.59715 | 3.1335 | 3.1346 | 3.13405 |
| Opadry II + wax | 2.4369 | 2.4239 | 2.4304 | 2.8742 | 2.8369 | 2.85555 |
| Opadry II | 2.3485 | 2.3285 | 2.3385 | 2.8058 | 2.776 | 2.7909 |
| Opadry II + wax | 2.2758 | 2.285 | 2.2804 | 2.6937 | 2.6768 | 2.68525 |
| Opadry AMB II | 2.2846 | 2.3045 | 2.29455 | 2.7431 | 2.7536 | 2.74835 |
| Opadry AMB II + wax | 2.2771 | 2.3081 | 2.2926 | 2.7213 | 2.7407 | 2.731 |

### Example 2: ALXN-1840 as an API

As shown in the table below, the ALXN-1840 tablet compound has a high level of sodium bicarbonate in the formulation to stabilize the API which is a chelator and highly reactive. Upon initial attempts to coat this compound the tablets would start disintegrating in the coating pan during the coating process. Applicants were able to successfully apply a film coating to the tablet after a wax coating was applied to the tablet.

After tablets were sufficiently heated, the exhaust was turned off and carnauba wax was slowly (over approximately 3-5 minutes) added to the pan sprinkling on top of the core tablets with the pan rotating. The tablets were tumbled with wax for 5 to 10 minutes at −3 RPM. The pan speed was adjusted based upon visual observation of the tablet bed. After the 5-10 minutes, the exhaust was turned on and run for an additional 5 to 7 minutes. The tablets could then be removed for film coating. OPADRY® 200 Clear 203A190001 was prepared as a 20% solids suspension and applied to a target tablet with weight gain of 20%. Next, Acryl-EZE White was prepared as a 20% solids suspension and applied to the film coated and wax coated tablet with weight gain of 35%. The following is the compositions of the tablet, first wax coating, and second film coating:

| | Formulation | |
|---|---|---|
| | Composition | Quantity |
| Component Name | (% w/w) | mg/tablet |
| WTX101 DRUG SUBSTANCE (ALXN1840) | 8.33 | 1.250 |
| (CSD AB)¹ | | |
| Sodium Bicarbonate, USP Grade 1 Powder | 24.99 | 3.749 |
| Microcrystalline cellulose (Avicel PH 112) | 65.93 | 9.890 |
| Sodium Stearyl Fumarate NF (Intragranular) | 0.25 | 0.038 |
| Sodium Stearyl Fumarate NF (Extragranular) | 0.50 | 0.075 |
| Tablet Core Total | 100 | 15.000 |
| Carnauba Wax Powdered #1 NF | 1.0 | 0.1500 |
| Opadry 200 Clear 203A190001 | 20.0 | 3.0300 |
| Purified Water USP/EP | n/a¹ | n/a¹ |
| Sub-Coated Tablet Total | 121 | 18.180 |
| Acryl-EZE White | 35.0 | 6.3630 |
| Purified Water USP/EP | n/a | n/a |
| Enteric Coated Tablet Total | 156 | 24.543 |

¹For informational purposes, API is red in color resulting in red - pink core minitablet. See photos in Figures.

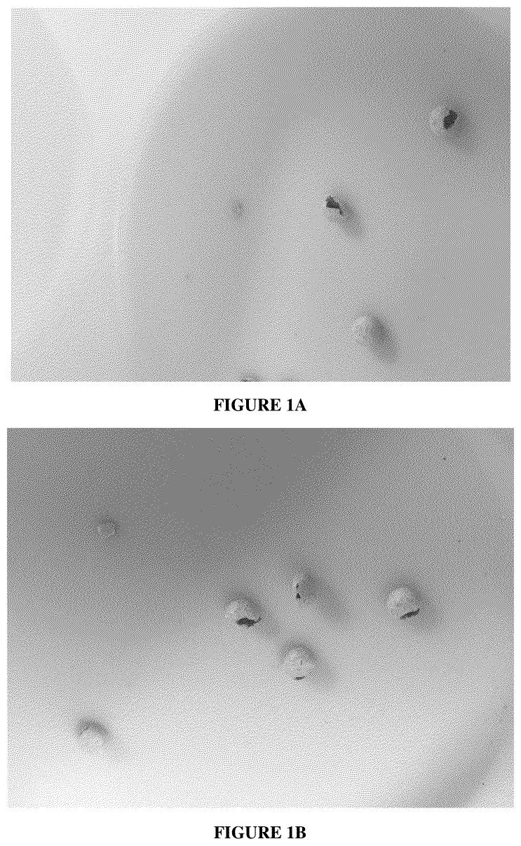

FIGS. 1A-1B illustrate minitablets subcoated without the wax addition. Specifically, these photos illustrate the result of coating the minitablets without wax. As seen in the photos, the minitablets were activated by the addition of water from the coating process. Activation of the minitablet core results in it being effervesced out of the coating shell during the coating process.

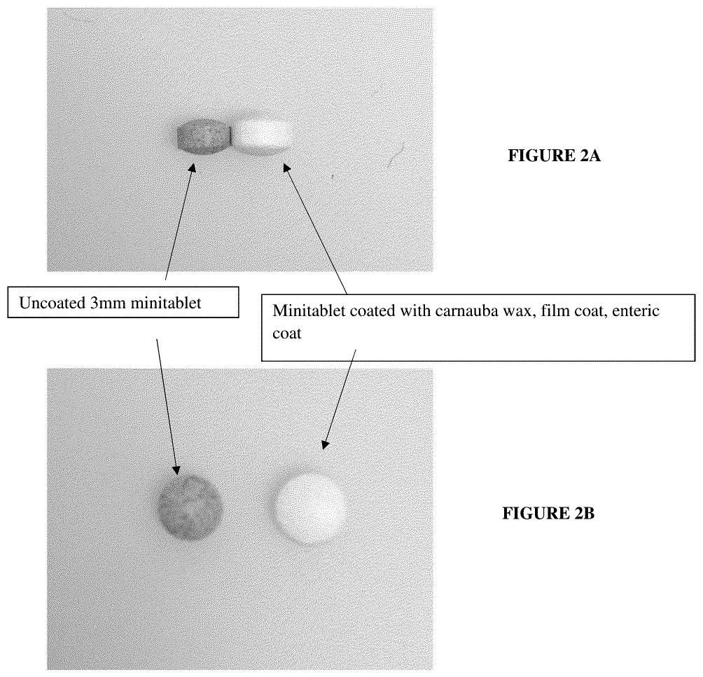

FIGS. 2A-2B illustrate uncoated core minitablet and enteric coated minitablets of Example 2 with 1% wax addition applied prior to the film and enteric coating. Specifically, the photos illustrate the uncoated core minitablet (red) prior to addition of wax and the fully coated white minitablet. The wax application prior to the film and enteric coating prevented the minitablet from becoming effervescent during the coating process. As such, Applicant discovered that the wax coating can provide initial protection from moisture which prevents the minitablets from activating during the initial application of the moisture barrier film coating.

### Additional Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a layer has a thickness of at least about 5 cm, about 10 cm, or about 15 cm is meant to mean that the layer has a thickness of at least about 5 cm, at least about 10 cm, or at least about 15 cm.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A pharmaceutical composition comprising:
a tablet comprising an active pharmaceutical ingredient (API);
a first coating on a surface of the tablet comprising wax; and
a second coating on a surface of the first coating, wherein the second coating comprises polyvinyl alcohol.

2. The pharmaceutical composition of claim 1, wherein the wax comprises carnauba wax.

3. The pharmaceutical composition of claim 1, wherein the pharmaceutical composition comprises 0.5-1.0% w/w wax.

4. The pharmaceutical composition of claim 1, wherein the first coating consists of wax.

5. The pharmaceutical composition of claim 1, wherein the second coating is an OPADRY® moisture barrier film coating.

6. The pharmaceutical composition of claim 1, further comprising a third coating on a surface of the second coating, wherein the third coating comprises an acrylic acid and/or an acrylate.

7. The pharmaceutical composition of claim 6, wherein the third coating is an ACRYL-EZE® enteric coating.

8. The pharmaceutical composition of claim 1, wherein the API comprises ALXN1840 or AKST4290.

9. The pharmaceutical composition of claim 1, wherein the tablet comprises sodium bicarbonate.

10. A method of preparing a pharmaceutical composition comprising:
compressing a tablet comprising an active pharmaceutical ingredient (API);
coating a surface of the tablet with a first coating comprising wax;
coating a surface of the first coating with a second coating comprising polyvinyl alcohol.

11. The method of claim 10, wherein coating the surface of the tablet with the first coating comprises coating the surface of the tablet such that the weight gain of the tablet with the first coating is 0.5-1.0% from the first coating.

12. The method of claim 10, wherein coating the surface of the first coating comprises coating the surface of the first coating such that the weight gain of the tablet and first coating is 1-30% from the second coating.

13. The method of claim 12, wherein the weight gain of the tablet and first coating from the second coating is 15-25%.

14. The method of claim 10, wherein the wax comprises carnauba wax.

15. The method of claim 10, wherein the first coating consists of wax.

16. The method of claim 10, wherein the second coating is an OPADRY® moisture barrier film coating.

17. The method of claim 10, further comprising coating a surface of the second coating with a third coating comprising an acrylic acid and/or an acrylate.

18. The method of claim 17, wherein the third coating is an ACRYL-EZE® enteric coating.

19. The method of claim 10, wherein the API comprises ALXN1840 or AKST4290.

20. The method of claim 10, wherein the tablet comprises sodium bicarbonate.
